(19) United States
(12) Patent Application Publication
(10) Pub. No.: US 2025/0228637 A1
(43) Pub. Date: Jul. 17, 2025

(54) ROBOTIC MEDICAL SYSTEM DRAPE ADAPTER ASSEMBLIES

(71) Applicant: EndoQuest Robotics, Inc., Houston, TX (US)

(72) Inventors: Hungu Kang, Gyeonggi-Do (KR); Jeihan Lee, Houston, TX (US); Jaesun Lee, Seongnam (KR); Bongjun Goh, Gyeonggi-Do (KR); Inseong Choe, Seoul (KR); Seungwoo Park, Houston, TX (US); Jiwon Choi, Houston, TX (US); Dongsuk Shin, Houston, TX (US)

(73) Assignee: EndoQuest Robotics, Inc., Houston, TX (US)

(21) Appl. No.: 18/415,502

(22) Filed: Jan. 17, 2024

(51) Int. Cl. A61B 46/10; A61B 17/00; A61B 34/20; A61B 34/30; A61B 46/23

(52) U.S. Cl. CPC A61B 46/10; A61B 34/20; A61B 34/30; A61B 46/23; A61B 2017/00477; A61B 2034/2074; A61B 2034/301

(57) ABSTRACT

A drape adapter assembly for a robotic medical system can include an outer housing configured to connect to the robotic medical system such that a drape is positioned between the outer housing and a portion of the robotic medical system, and an inner housing disposed at least partially within the outer housing and configured to carry one or more moveable actuation elements therein. The inner housing can include a first inner housing portion and a second inner housing portion fastened to the first inner housing portion such that the first inner housing portion and the second inner housing portion are axially retained to the outer housing.

### FIELD

This disclosure relates to robotic medical system drape adapter assemblies.

### BACKGROUND

Minimally invasive surgery such as endoluminal and single-site robotic surgery offer significant advantages versus traditional robotic surgery. For example, in endoluminal robotic surgery, no incision need be made to access difficult to access locations within a patient's natural lumen. This dramatically reduces and/or eliminates recovery time and improves procedural safety. A single-site system reduces incisions to a minimum single-site, which reduces an otherwise larger number of incisions to provide access for certain procedures.

Certain endoluminal and single-site robotic surgical systems have been proposed. Examples of such systems and related components can be found in U.S. Pat. No. 10,881,422, as well as U.S. Patent Application Publication Nos. US20210322046, US20210322045, US20190117247, US20210275266, US20210267702, US20200107898, US20200397457, US20200397456, US20200315645, and PCT Publication No. WO2023/101974, all of the above being incorporated by reference herein in their entirety.

Robotic surgical systems can include drape adapter assemblies for providing a sterile area within the drape while connecting to one or more mechanical components of the robotic surgical system that are outside of the sterile area. There is still a need in the art for improvements over traditional assemblies.

Conventional surgical robotics and systems have generally been considered satisfactory for their intended purpose. However, there is still a need in the art for improved robotic surgical systems, devices, methods, controls, and components, especially those configured for endoluminal and single-site surgery. The present disclosure provides improvements in such areas, for example.

### SUMMARY

In accordance with at least one aspect of this disclosure, a drape adapter assembly for a robotic medical system can include an outer housing configured to connect to the robotic medical system such that a drape is positioned between the outer housing and a portion of the robotic medical system, and an inner housing disposed at least partially within the outer housing and configured to carry one or more moveable actuation elements therein. The inner housing can include a first inner housing portion and a second inner housing portion fastened to the first inner housing portion such that the first inner housing portion and the second inner housing portion are axially retained to the outer housing.

The outer housing can include an inner wall and one or more tabs extending from the inner wall. The one or more tabs can be configured to be disposed between the first inner housing portion and the second inner housing portion to axially retain the inner housing. The first inner housing portion can be configured to be inserted into the outer housing through a first outer housing opening and the second inner housing portion can be configured to be inserted into the outer housing through a second outer housing opening (e.g., opposite the first outer housing opening) such that the one or more tabs of the outer housing are trapped between the first inner housing portion and the second inner housing portion.

In certain embodiments, the inner housing can define a trough. The one or more tabs of the outer housing can radially extend into the trough. The inner housing can be configured to rotate relative to the outer housing between an instrument-retained position and instrument-unrestrained position.

The one or more tabs can include one or more interaction tabs. The inner housing can include one or more interaction components configured to interact with the one or more interaction tabs. The one or more interaction tabs and the one or more interaction components can be configured to retain the inner housing in the instrument-unrestrained position and the instrument-retained position, and/or can be configured to provide tactile feedback when the inner housing is rotated to the instrument-retained position or instrument-unrestrained position.

The one or more interaction tabs can include a first radial detent and a second radial detent. The one or more interaction components can include a biased interaction member (e.g., a ball) biased radially toward (e.g., radially outwardly) the one or more interaction tabs to radially engage the biased interaction member to the one or more interaction tabs and to cause the biased interaction member to enter the first radial detent or the second radial detent when rotationally aligned therewith. The biased interaction member and the first and second radial detents can be configured to allow the biased interaction member to be urged out of the first and second radial detents with a relative rotation (e.g., of suitably high force to overcome the bias) of the outer housing relative to the inner housing.

In certain embodiments, the one or more tabs can include one or more limit tabs, wherein the inner housing includes one or more limit features configured to interact with the one or more limit tabs to limit a range of rotational motion of the inner housing relative to the outer housing. The one or more limit tabs or the one or more limit features can include a limit pin extending axially therefrom, and the other of the one or more limit tabs and the one or more limit features can include an axial slot configured to receive the limit pin. A dimension of the axial slot can define the range of rotation motion of the inner housing relative to the outer housing.

The inner housing can define one or more axial channels configured to receive and allow axial movement of the actuation elements. The outer housing can include one or more instrument alignment tabs distal of the one or more tabs that extend radially inward from the inner wall of the outer housing. The one or more instrument alignment tabs can be configured to align an instrument for insertion in the instrument-unrestrained position, and to retain an instrument to the assembly in the instrument-retained position. The first inner housing portion can include one or more alignment slots defined in an outer surface thereof configured to align with the instrument alignment tabs to allow axial advancement of the first inner housing portion into the outer housing from the first outer housing opening.

The first inner housing portion can include one or more axial instrument alignment features configured to force rotational alignment of the instrument and the inner housing when the instrument is inserted in the instrument-unrestrained position. The one or more axial alignment features can include one or more axial pins extending distally from the first inner housing portion and configured to insert into an alignment aperture on a proximal side of the instrument.

In certain embodiments, the inner housing can be entirely contained within the outer housing. The outer housing can be generally cylindrical. The outer housing can also have a proximal portion that has a bell shape. In certain embodiments, the outer housing can include one or more alignment lands extending from an inner wall of the proximal portion for alignment with an instrument controller of the robotic medical system.

In accordance with at least one aspect of this disclosure, a drape adapter for an instrument controller of a robotic medical system can include an outer housing, and an inner housing axially retained to the outer housing and configured to rotate relative to outer housing within a limited range to move between an instrument-unrestrained position and an instrument-retained position. The outer housing can be configured to axially install to the instrument controller, and the inner housing can be configured to rotationally align one or more actuation elements of the inner housing at a proximal side of the inner housing with one or more corresponding actuators of the instrument controller such that axial movement of the one or more corresponding actuators of the instrument controllers causes respective axial movement of the one or more actuation elements.

A robotic surgical instrument controller assembly can include an instrument controller having a mounting interface, an outer housing is detachably mountable to at least a portion of the mounting interface, and an inner housing axially retained to the outer housing and is rotatable relative to outer housing within a limited range to move between an instrument-unrestrained position and an instrument-retained position. The outer housing can be configured to axially install to the instrument controller, and the inner housing is configured to rotationally align one or more actuation elements of the inner housing at a proximal side of the inner housing with one or more corresponding actuators of the instrument controller such that axial movement of the one or more corresponding actuators of the instrument controllers causes respective axial movement of the one or more actuation elements. The assembly can further include a drape ring detachably mountable to at least a portion of the mounting interface.

These and other features of the embodiments of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, embodiments thereof will be described in detail herein below with reference to certain figures, wherein:

FIG. 1A is a distal perspective view of an embodiment of a drape adapter assembly in accordance with this disclosure.

FIG. 1B is a proximal perspective view of the embodiment of FIG. 1A.

FIG. 2A is an exploded view of the embodiment of FIG. 1A, showing an outer housing and an inner housing, the inner housing having a first inner housing portion and a second inner housing portion and other components separated.

FIG. 2B is an exploded view of the embodiment of FIG. 2A, showing actuation elements axially placed within the second inner housing portion.

FIG. 2C is an exploded view of the embodiment of FIG. 2B, showing actuation element guide pins radially inserted into the second inner housing portion.

FIG. 2D is an exploded view of the embodiment of FIG. 2C, showing the second inner housing portion inserted into the outer housing, wherein the outer housing is shown cutaway or in phantom for clarity.

FIG. 2E is an exploded view of the embodiment of FIG. 2D, showing the first inner housing portion inserted into the outer housing.

FIG. 2F is an exploded view of the embodiment of FIG. 2E, showing fasteners installed to fix the first inner housing portion and the second inner housing portion together within the outer housing and such that the inner housing is axially trapped within the outer housing.

FIG. 3A is a perspective view of the outer housing of the embodiment of FIG. 1A.

FIG. 3B is a distal elevation view of the embodiment of FIG. 3A.

FIG. 3C is a proximal elevation view of the embodiment of FIG. 3A.

FIG. 3D is a cross-sectional view of the embodiment of FIG. 3A.

FIG. 4A is a perspective view of the inner housing of the embodiment of FIG. 1A.

FIG. 4B is a side elevation view of the embodiment of FIG. 4A, showing an embodiment of a biased interaction member.

FIG. 4C is a perspective view of the embodiment of FIG. 4A, showing an axial slot and illustrating an outer housing limit pin in position.

FIG. 4D is a perspective cross-sectional view of the embodiment of FIG. 4A, showing a cross-section taken axially and orthogonal to the view of FIG. 4B.

FIG. 4E is a cross-sectional view of the embodiment of FIG. 4A, showing a cross-section taken radially and orthogonal to the view of FIG. 4B.

FIG. 4F is a perspective cross-sectional view of the embodiment of FIG. 4A, showing a cross-section taken proximal to the view of FIG. 4E.

FIG. 5A is a perspective view of the embodiment of FIG. 1A, shown in an instrument-unrestrained position and showing the outer housing in phantom to illustrate a relative position of the inner housing to the outer housing.

FIG. 5B is a cross-sectional view of the embodiment of FIG. 5A, showing a relative position of the inner housing relative to the outer housing in the instrument-unrestrained position.

FIG. 5C is a close-up partial cross-sectional view of the embodiment of FIG. 5A, illustrating a relative position of a biased interaction member of the inner housing relative to an interaction tab of the outer housing in the instrument-unrestrained position.

FIG. 5D is a close-up partial perspective view of the embodiment of FIG. 5A, showing the outer housing in phantom to illustrate a relative position of the biased interaction member of the inner housing relative to the interaction tab of the outer housing in the instrument-unrestrained position.

FIG. 5E is a close-up partial perspective view of the embodiment of FIG. 5A, showing the outer housing in phantom to illustrate a relative position of the limit pin of the outer housing relative to the axial slot of the outer inner housing in the instrument-unrestrained position.

FIG. 6A is a perspective view of the embodiment of FIG. 1A, shown in an instrument-retained position and showing the outer housing in phantom to illustrate a relative position of the inner housing to the outer housing.

FIG. 6B is a cross-sectional view of the embodiment of FIG. 6A, showing a relative position of the inner housing relative to the outer housing in the instrument-retained position.

FIG. 6C is a close-up partial cross-sectional view of the embodiment of FIG. 6A, illustrating a relative position of a biased interaction member of the inner housing relative to an interaction tab of the outer housing in the instrument-retained position.

FIG. 6D is a close-up partial perspective view of the embodiment of FIG. 6A, showing the outer housing in phantom to illustrate a relative position of the biased interaction member of the inner housing relative to the interaction tab of the outer housing in the instrument-retained position.

FIG. 6E is a close-up partial perspective view of the embodiment of FIG. 6A, showing the outer housing in phantom to illustrate a relative position of the limit pin of the outer housing relative to the axial slot of the outer inner housing in the instrument-retained position.

FIG. 7A is a partial perspective view of an instrument release assembly associated with the outer housing in accordance with this disclosure.

FIG. 7B illustrates the instrument release assembly of FIG. 7A, shown having a slider button in phantom and in an instrument-retained position.

FIG. 7C illustrates the instrument release assembly of FIG. 7A, shown having a slider button in phantom and in an instrument-released position.

FIG. 8A shows a partial perspective cross-sectional view of a proximal portion of the outer housing of FIG. 1A, showing an instrument controller attachment assembly in accordance with this disclosure, showing a push button configured to move radially outward guided by axial pins.

FIG. 8B shows another partial perspective cross-sectional view of a proximal portion shown in FIG. 8A further illustrating the instrument controller attachment assembly.

FIG. 9A is a partial perspective view of an embodiment of an proximal instrument hub in accordance with this disclosure.

FIG. 9B is a partial perspective view of an embodiment of an proximal instrument hub in accordance with this disclosure.

FIG. 10A is a partial perspective view of an embodiment of an instrument controller in accordance with this disclosure.

FIG. 10B is a partial perspective view of an embodiment of an instrument controller cover in accordance with this disclosure.

FIG. 11A is a perspective view of an embodiment of an instrument controller in accordance with this disclosure.

FIG. 11B is a perspective view of the embodiment of an instrument controller of FIG. 11A.

FIG. 11C is a perspective view illustrating a drape ring relative to the embodiment of FIG. 11A.

FIG. 11D is a perspective view illustrating the drape ring of FIG. 11C installed over the embodiment of FIG. 11A.

### DETAILED DESCRIPTION

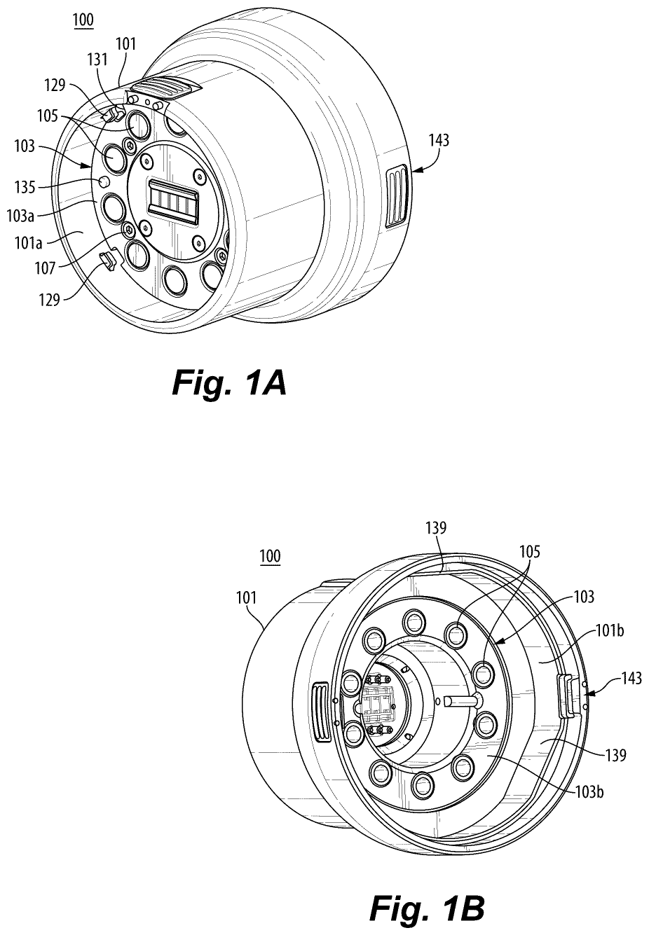

Reference will now be made to the drawings wherein like reference numerals identify similar structural features or aspects of the subject disclosure. For purposes of explanation and illustration, and not limitation, an illustrative view of an embodiment of a drape adapter assembly in accordance with the disclosure is shown in FIGS. 1A and 1s designated generally by reference character 100. Other views, embodiments, and/or aspects of this disclosure are illustrated in FIGS. 1B-11D.

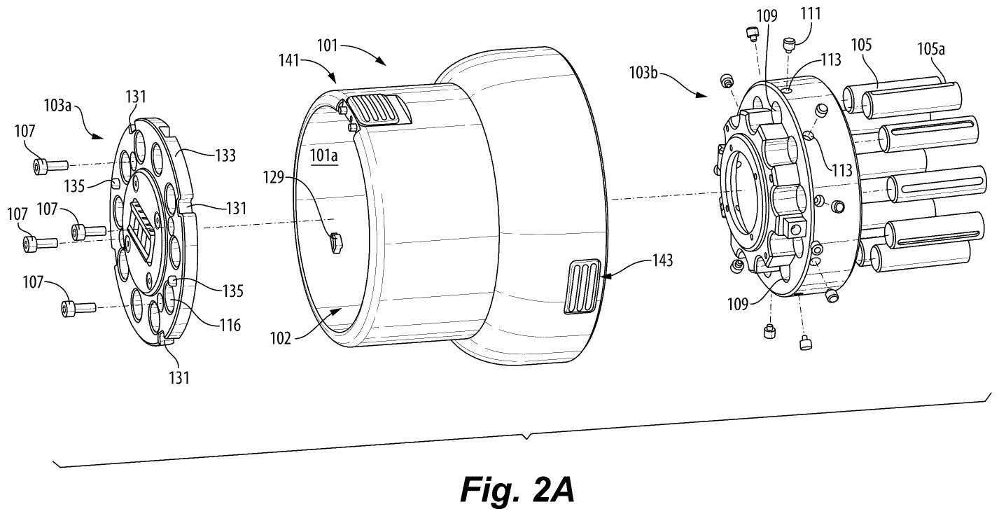

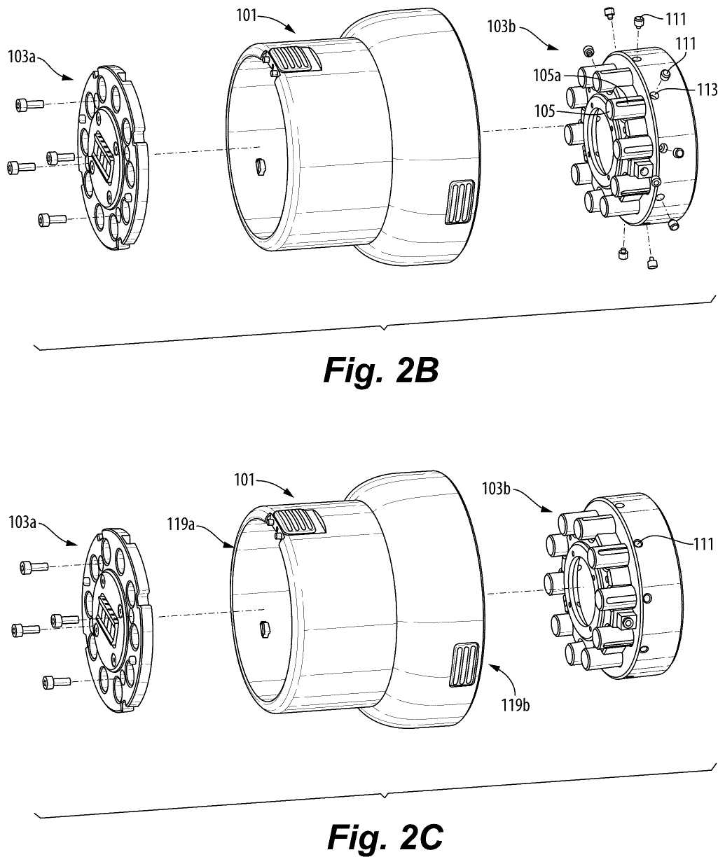

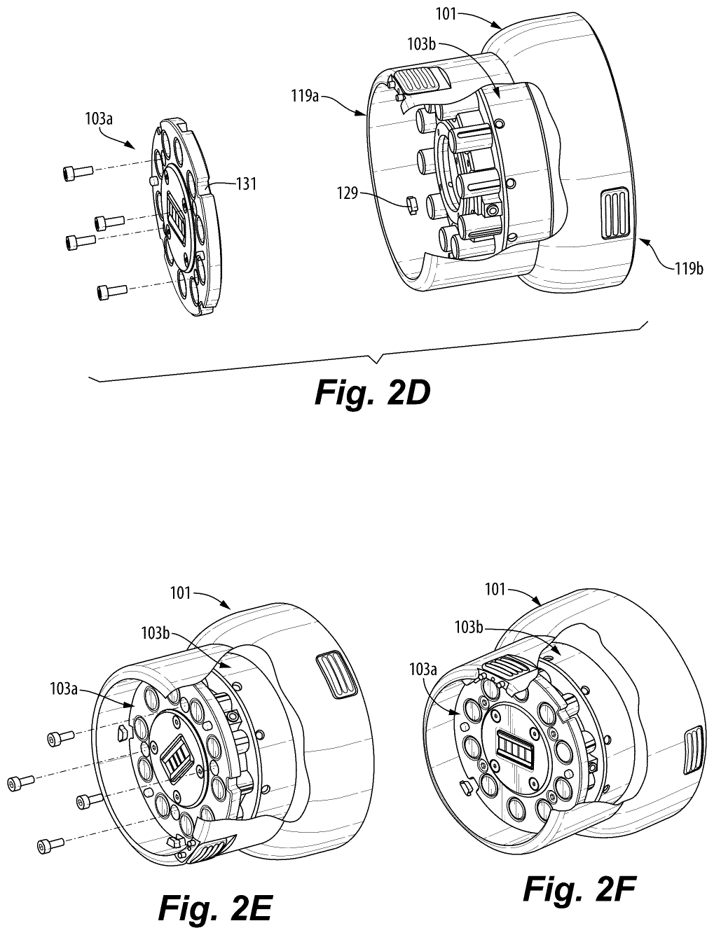

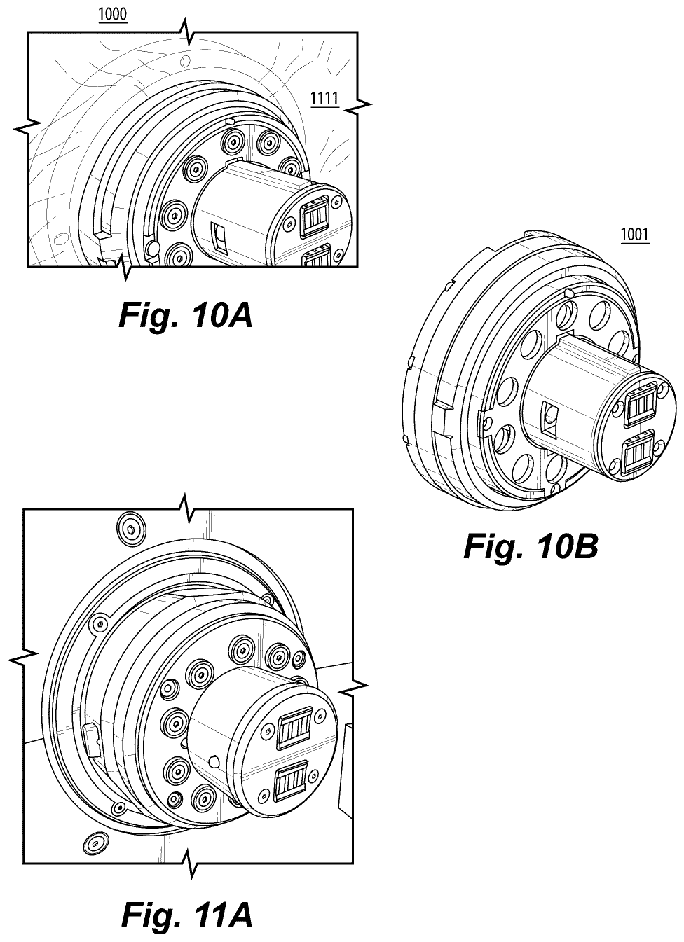

In accordance with at least one aspect of this disclosure, referring to FIGS. 1A and 1B, an embodiment of a drape adapter assembly 100 for a robotic medical system (not shown) is shown. In certain embodiments, e.g., as shown, the assembly 100 includes an outer housing 101 configured to connect to the robotic medical system such that a drape (e.g., drape 1111 shown in FIG. 10A) is positioned between the outer housing 101 and a portion of the robotic medical system (e.g., an instrument controller thereof). The outer housing 101 defines an interior space 102, e.g., as shown in FIG. 2A. In certain embodiments, e.g., as shown, the assembly 100 also includes an inner housing 103 disposed at least partially within the interior space 102 of the outer housing 101 and configured to carry one or more moveable actuation elements 105 therein (e.g., axially sliding pins or posts within an axial channel 109). The robotic medical system can include any suitable robotic medical system, e.g., as described in commonly owned International Patent Application Publication No. WO2023/101971, as well as U.S. patent application Ser. No. 18/198,761, filed May 17, 2023, and U.S. Patent Application 63/284,289 filed Nov. 30, 2021, and any other references incorporated therein, all of which are incorporated by reference herein in their entirety.

Referring additionally to FIG. 2A, In certain embodiments, e.g., as shown, the inner housing 103 includes a first inner housing portion 103a (e.g., a distal portion as shown) and a second inner housing portion 103b (e.g., a proximal portion as shown). In certain embodiments, the first inner housing portion 103a and a second inner housing portion 103b can be integrally formed as one piece. In other embodiments, the second inner housing portion 103b can be fixedly adjacent (e.g., fastened, screwed on, glued, magnetically attached, otherwise attached) to the first inner housing portion 103a (e.g., via one or more fasteners 107 such as, e.g., axial screws) such that the first inner housing portion 103a and the second inner housing portion 103b are axially retained to the outer housing 101. FIGS. 2B-2F collectively illustrate the embodiment of FIG. 1A being progressively assembled in accordance with an embodiment of a method of assembly in accordance with this disclosure. For example, as shown in FIG. 2B, the actuation elements 105 can be inserted into axial channels 109 of the second inner housing portion 103b. As shown in FIG. 2C, the actuation elements 105 can be pinned (e.g., in a slot 105a thereof) by radial pins 111 inserted into radial holes 113 (e.g., press fit, threaded, or otherwise radially retain) to be constrained to axial sliding motion within the axial channels 109. As shown in FIG. 2D, the assembled second inner housing portion 103b can be inserted into a proximal opening of the outer housing 101. As shown in FIG. 2E, first inner housing portion 103a is inserted into the outer housing 101 via a distal opening of the outer housing 101 to abut the second inner housing portion 103b. As shown in FIG. 2F, the first inner housing portion 103a and the second inner housing portion 103b can be fastened together using one or more fasteners 107 (e.g., axial screws as shown).

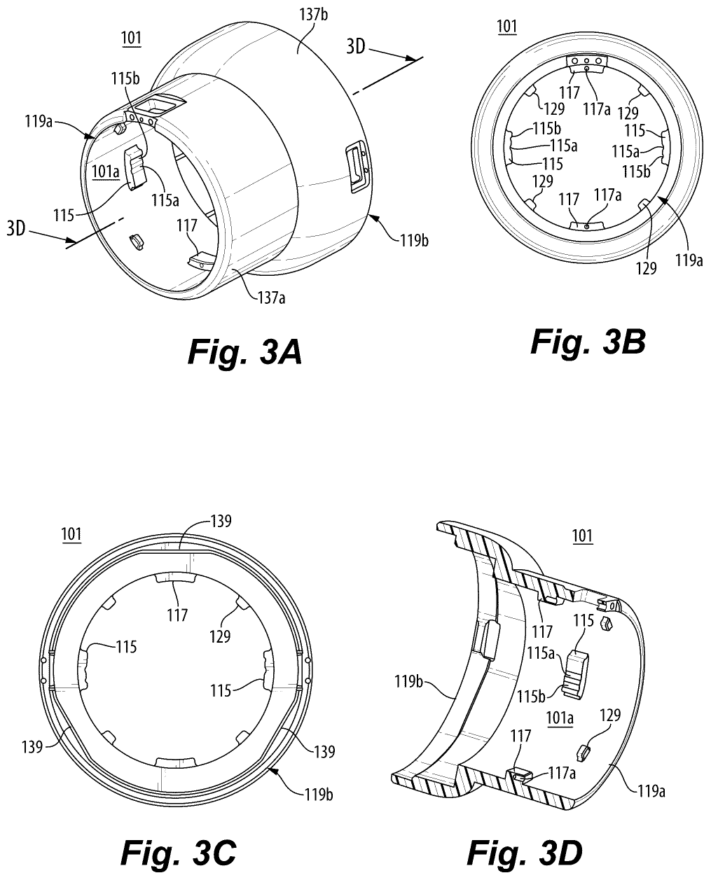

Referring additionally to FIGS. 3A-3D, in certain embodiments, e.g., as shown, the outer housing 101 includes an inner wall 101a and one or more tabs 115, 117 (e.g., guide tabs) extending from the inner wall 101a. The one or more tabs 115, 117 can be configured to be disposed between the first inner housing portion 103a and the second inner housing portion 103b to axially retain the inner housing 103. For example, the first inner housing portion 103a can be configured to be inserted into the outer housing 101 through a first outer housing opening 119a (e.g., the distal opening as shown in FIGS. 2D and 2E) and the second inner housing portion 103b can be configured to be inserted into the outer housing 101 through a second outer housing opening 119b (e.g., opposite the first outer housing opening 119a; e.g., a proximal opening as shown in FIGS. 2C and 2D) such that the one or more tabs 115, 117 of the outer housing 101 are trapped between the first inner housing portion 103a and the second inner housing portion 103b.

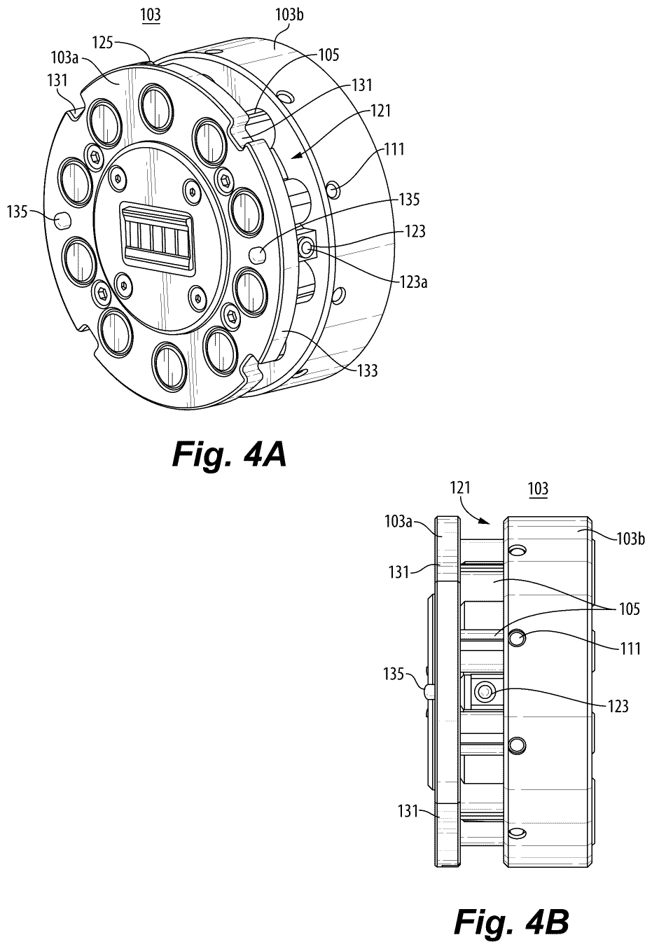

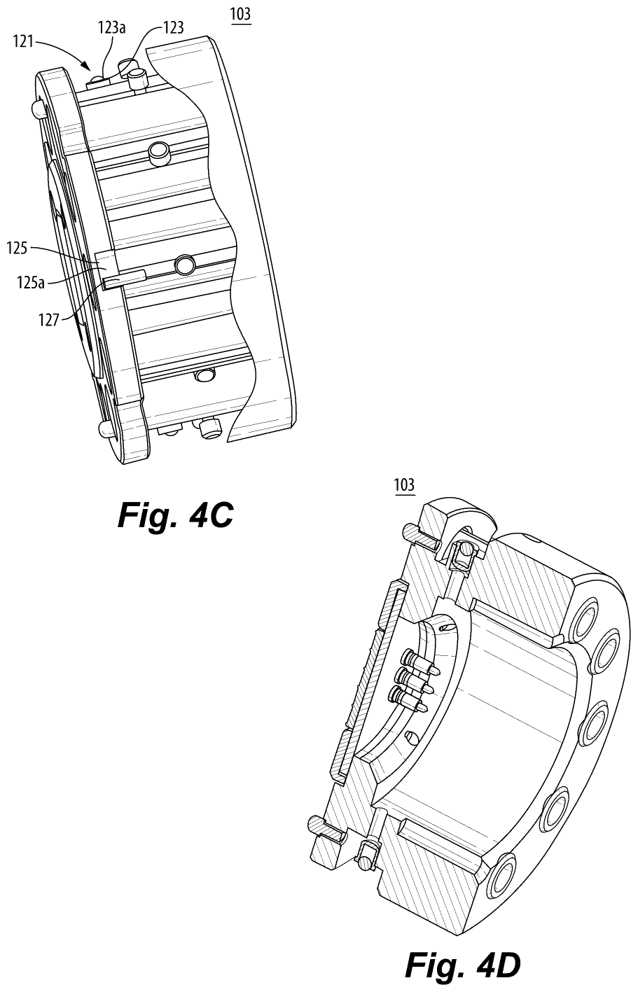

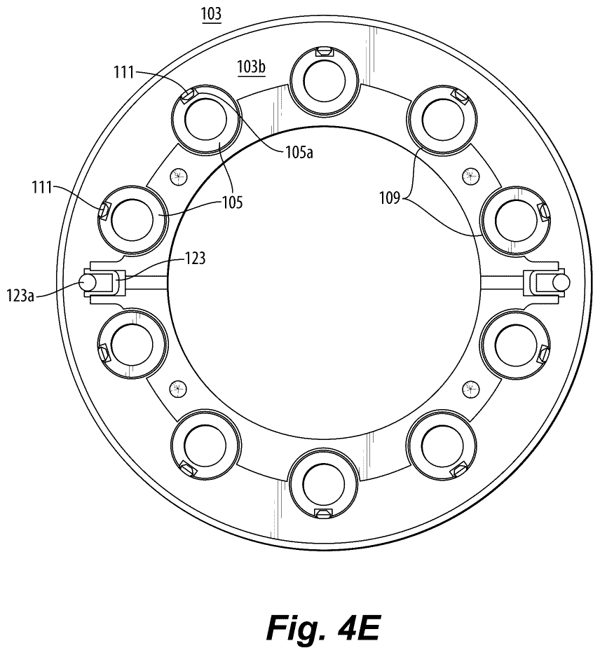

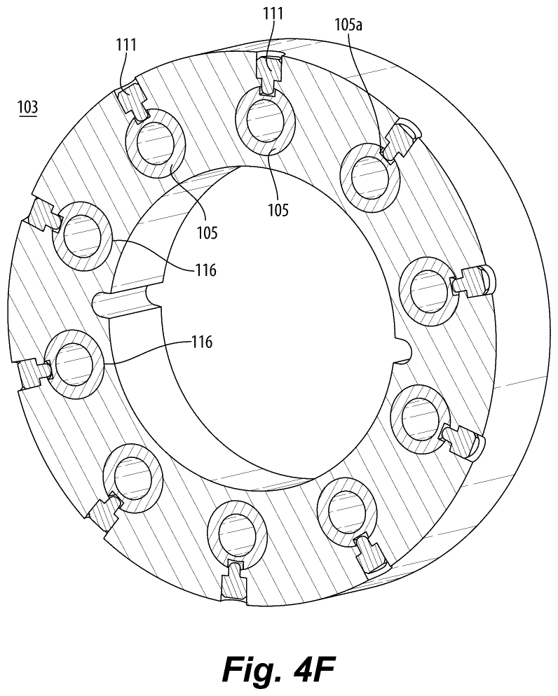

In certain embodiments, for example, referring additionally to FIGS. 4A-4F, the inner housing 103 can define a trough 121. The one or more tabs 115, 117 of the outer housing 101 can radially extend into the trough 121. The inner housing 103 can be configured to rotate relative to the outer housing 101 (e.g., guided by the one or more tabs 115, 117) while being axially retained by the one or more tabs 115, 117. For example, the one or more tabs 115, 117 may be smaller in axial thickness than the trough 121 to allow free rotation while preventing substantial axial movement of the inner housing. However, the fit of the inner housing 103 around the one or more tabs 115, 117 can be as tight as desired (e.g., contacting and/or in compression) to provide any suitable resistance. The inner housing 103 can be configured to rotate relative to the outer housing 101 between an instrument-retained position and instrument-unrestrained position.

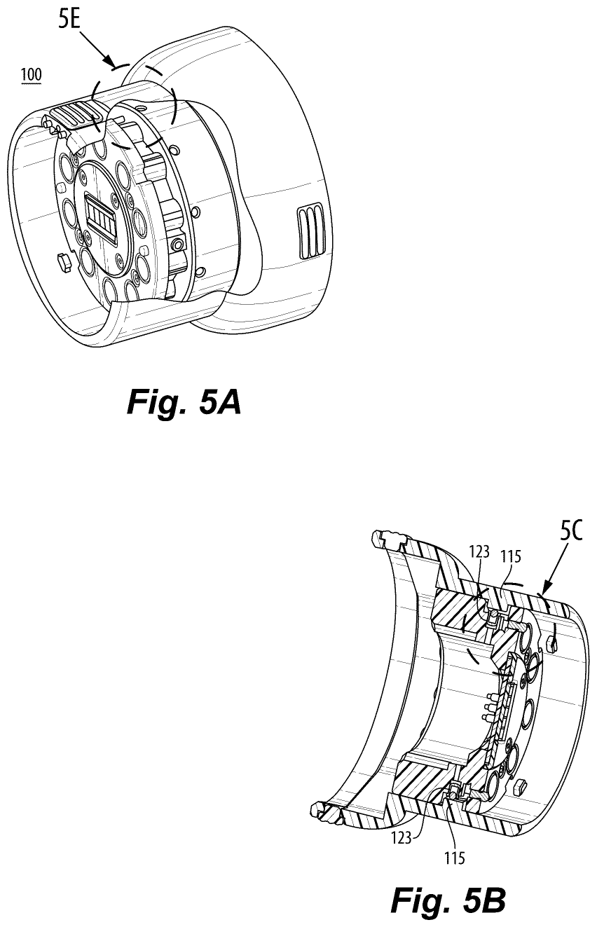

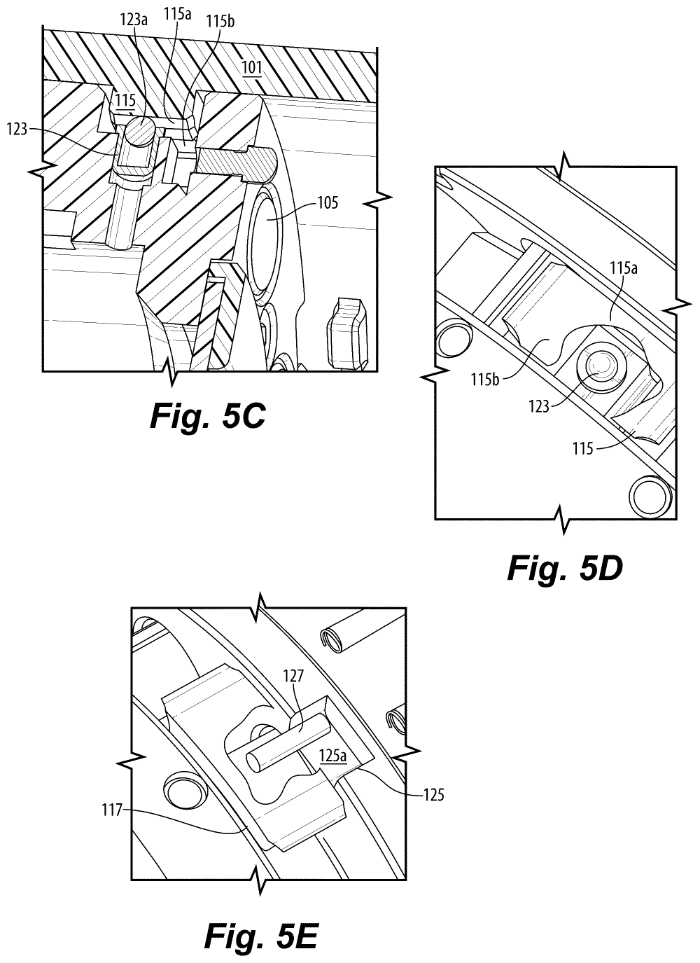

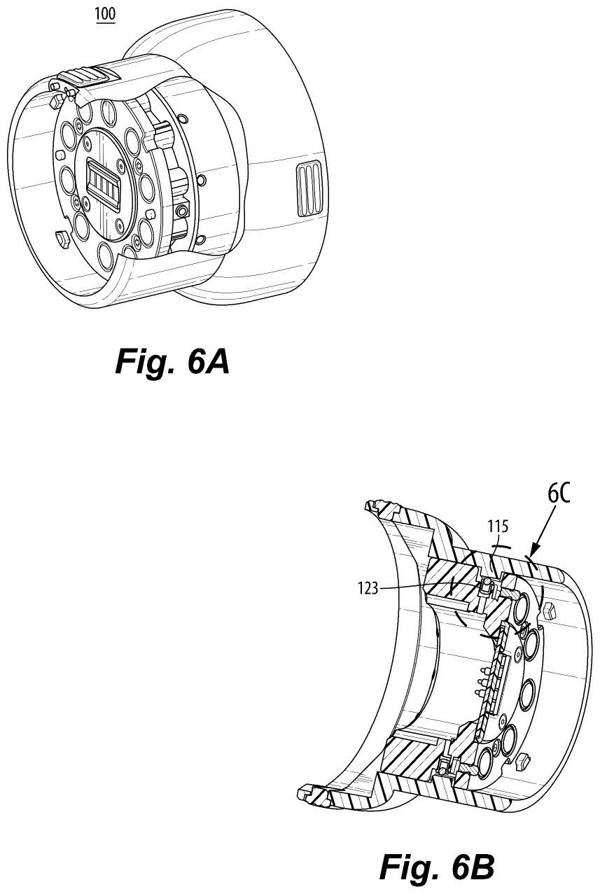

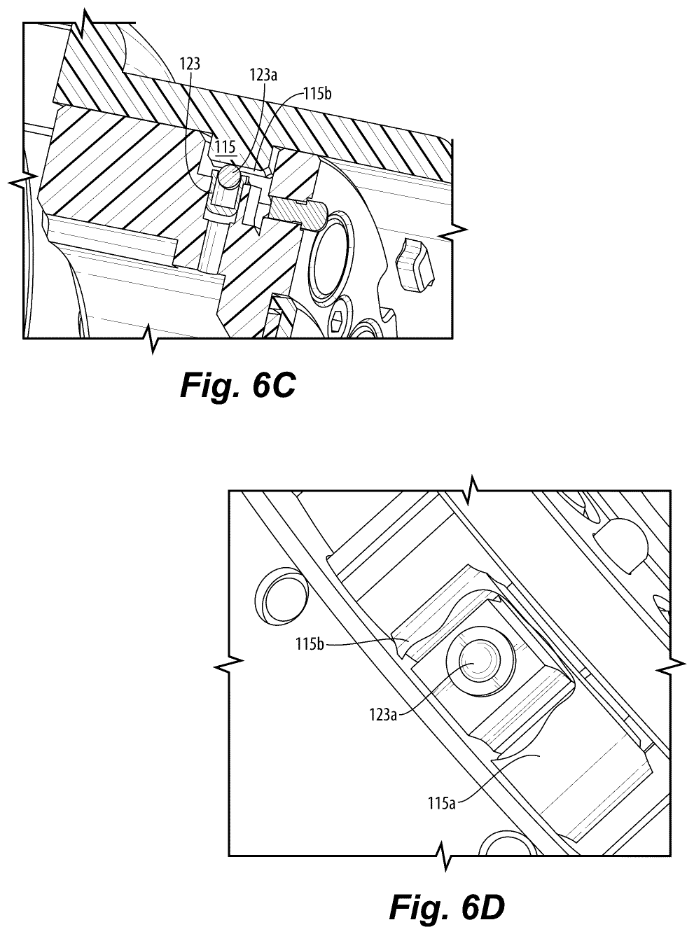

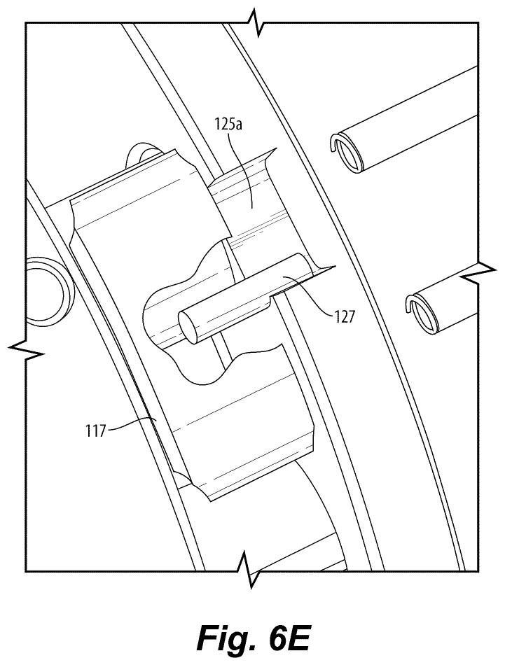

In certain embodiments, e.g., as shown, the one or more tabs 115, 117 of the outer housing 101 include one or more interaction tabs 115. The inner housing 103 can include one or more interaction components 123 (e.g., two in diametrically opposed locations within the trough 121) configured to interact (e.g., engage) with the one or more interaction tabs 115. In certain embodiments, the one or more interaction tabs 115 and the one or more interaction components 123 can be configured to retain the inner housing 103 in the instrument-unrestrained position (e.g., as shown in FIGS. 5A-5E) and the instrument-retained position (e.g., as shown in FIGS. 6A-6E), e.g., until forced to move out of position with a suitable force to overcome the bias. In certain embodiments, additional or alternatively, the one or more interaction tabs 115 and the one or more interaction components 123 can be configured to provide tactile feedback when the inner housing 103 is rotated to the instrument-retained position or instrument-unrestrained position.

In certain embodiments, e.g., as shown, the one or more interaction tabs 115 include a first radial detent 115a (e.g., corresponding to the instrument-unrestrained position) and a second radial detent 115b (e.g., corresponding to the instrument-retained position). The one or more interaction components 123 can include a biased interaction member 123a (e.g., a spring loaded ball disposed on the second inner housing portion 103b) biased radially toward (e.g., radially outwardly) the one or more interaction tabs 115 to radially engage the biased interaction member 123a to the one or more interaction tabs 115 and to cause the biased interaction member 123a to enter the first radial detent 115a or the second radial detent 115b when rotationally aligned therewith. The biased interaction member 123a and the first and second radial detents 115a, 115b can be configured (e.g., shaped such as curved, slanted, etc.) to allow the biased interaction member 123a to be urged out of the first and second radial detents 115a, 115b with a relative rotation (e.g., of suitably high force to overcome the bias of biased interaction member 123a) of the outer housing relative 101 to the inner housing 103. It is contemplated that the reverse arrangement can be utilized such that the one or more interaction tabs 115 can include the biased interaction member 123a and the one or more interaction components 123 can include the first and second radial detents 115a, 115b. Certain embodiments can include other suitable motion resistive arrangements that allow for one or more (e.g., two) discreet positions to be rotationally selected (e.g., clicking into place) is contemplated herein.

In certain embodiments, e.g., as shown, the one or more tabs 115, 117 include one or more limit tabs 117. The inner housing 103 can include one or more limit features 125 configured to interact with the one or more limit tabs 117 to limit a range of rotational motion of the inner housing 103 relative to the outer housing 101 (e.g., between the instrument-unrestrained position and the instrument-retained position). The one or more limit tabs 117 or the one or more limit features 125 can be or include a limit pin 127 extending axially therefrom, and the other of the one or more limit tabs 117 and the one or more limit features 125 can include an axial slot 125a configured to receive the limit pin 127. For example, the axial slot 125a can be defined partially axially (partly through the thickness) and circumferentially on an outer surface 133 of the first inner housing portion 103a. A dimension of the axial slot 125a can define the range of rotation motion of the inner housing 103 relative to the outer housing 101, for example. In certain embodiments, the one or more (e.g., two) axial slots 125a can allow a range of about 0 to about 7 degrees of rotation, for example. In certain embodiments, the one or more limit tabs 117 can have a range of about 0 to about 7 degrees of rotation between the first and second radial detents 115a, 115b.

While it is shown that the one or more limit tabs 117 include an axial hole 117a (as shown in FIG. 3D) having the limit pin 127 disposed therein (e.g., press fit or otherwise disposed), it is contemplated that the one or more limit tabs 117 can include a slot (e.g., axially and circumferentially defined similar to slot 125a as shown) configured to receive the limit pin 127, and the limit pin 127 or other suitable axial protrusion can extend from the first inner housing portion 103 into the slot in the one or more limit tabs 117. Certain embodiments can include other suitable arrangements for limiting relative motion between the inner housing 103 and the outer housing 101.

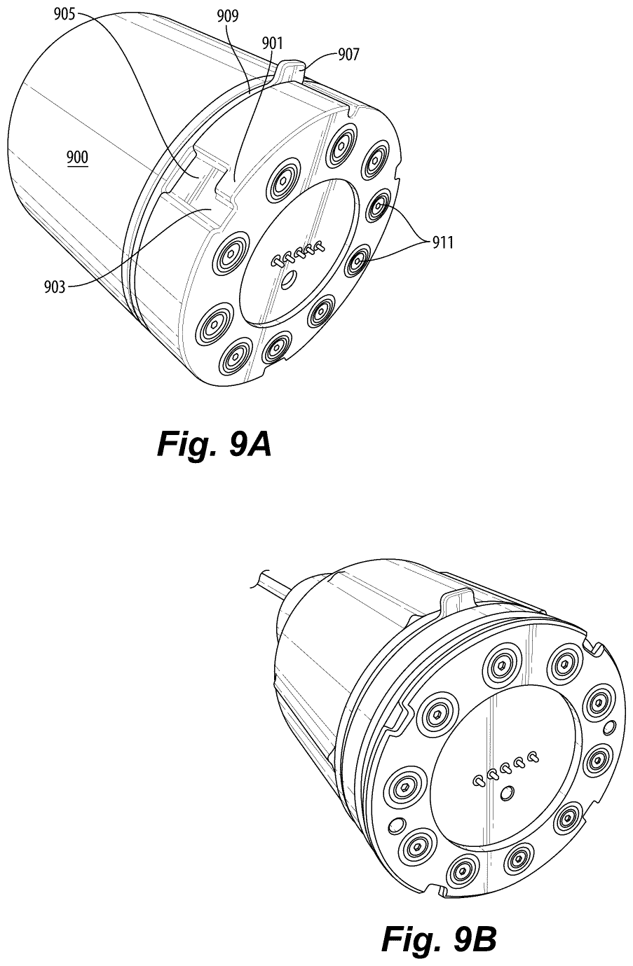

As disclosed above, in certain embodiments, e.g., as shown, the inner housing 103 defines one or more axial channels 109 configured to receive and allow axial movement of the actuation elements 105. In certain embodiments, e.g., as shown, the outer housing 101 includes one or more instrument alignment tabs 129 distal of the one or more tabs 115, 117 that extend radially inward from the inner wall 101a of the outer housing 101. The one or more instrument alignment tabs 129 can be configured to align an instrument for insertion in the instrument-unrestrained position, and to retain an instrument to the assembly 100 in the instrument-retained position. For example, a proximal end of the instrument can include one or more hub tabs 901 or hub tab walls 901b (e.g., as shown in FIG. 9A or FIG. 9B) that can be retained and/or compressed between the instrument alignment tabs 129 and the first inner housing portion 103a when rotated from the instrument-unrestrained position to the instrument-retained position. In the instrument-unrestrained position (e.g., as shown in FIGS. 5A-5E), the hub tabs of the instrument can be advanced past the instrument alignment tabs and also cause the actuation elements 105 to align with a proximal face of actuators of the instrument (e.g., for pushing only engagement). The instrument and the inner housing 103 can be rotated together to the instrument-retained position (e.g., as shown in FIGS. 6A-6E), and thereby can cause the hub tabs to rotate behind the instrument alignment tabs 129 to be axially retained between the instrument alignment tabs and the first inner housing portion 103a while also maintaining alignment of the actuation elements 105 with the proximal face of the actuators of the instrument (e.g., for pushing only engagement).

The first inner housing portion 103a can include one or more alignment slots 131 defined in an outer surface 133 (e.g., a radial outer diameter) thereof configured to align with the instrument alignment tabs 129 to allow axial advancement of the first inner housing portion 103 into the outer housing 101 from the first outer housing opening 119a. Embodiments can be configured to allow for the first inner housing portion 103a to sandwich the one or more tabs 115, 117 with the second inner housing portion 103b, and also define a suitable distance between the instrument alignment tabs 129 and the distal face of the first inner housing portion 103a to allow hub tabs of a proximal portion of an instrument to fit therebetween.

In certain embodiments, the first inner housing portion 103a can include one or more axial instrument alignment features 135 configured to force rotational alignment of the instrument and the inner housing 103 when the instrument is inserted in the instrument-unrestrained position. The one or more axial alignment features 135 can include one or more axial pins (e.g., as shown in 8A) extending distally from the first inner housing portion 103a and configured to insert into an alignment aperture (not shown) on a proximal side of the instrument. Such features can ensure rotational alignment between the inner housing 103 and the instrument to ensure proper alignment of the actuation elements 105 with the actuators of the instrument.

In certain embodiments, the inner housing 103 can be entirely contained within the outer housing 101 (e.g., as shown). The outer housing 101 can be generally cylindrical, for example, e.g., as shown. The outer housing 101 can include a distal portion 137a and a proximal portion 137b that has a bell shape (e.g., and is wider in diameter than the distal portion 137a). In certain embodiments, the outer housing 101 can include one or more alignment lands 139 extending from an inner wall 101b of the proximal portion 137b for alignment with an instrument controller of the robotic medical system.

In accordance with at least one aspect of this disclosure, a drape adapter (e.g., assembly 100) for an instrument controller of a robotic medical system can include an outer housing 101, and an inner housing 103 axially retained to the outer housing 101 and configured to rotate relative to outer housing 101 within a limited range to move between an instrument-unrestrained position and an instrument-retained position. The outer housing 101 can be configured to axially install to the instrument controller of the robotic medical system, and the inner housing can be configured to rotationally align one or more actuation elements 105 of the inner housing 103 at a proximal side of the inner housing 103 with one or more corresponding actuators of the instrument controller such that axial movement of the one or more corresponding actuators of the instrument controllers causes respective axial movement of the one or more actuation elements 105.

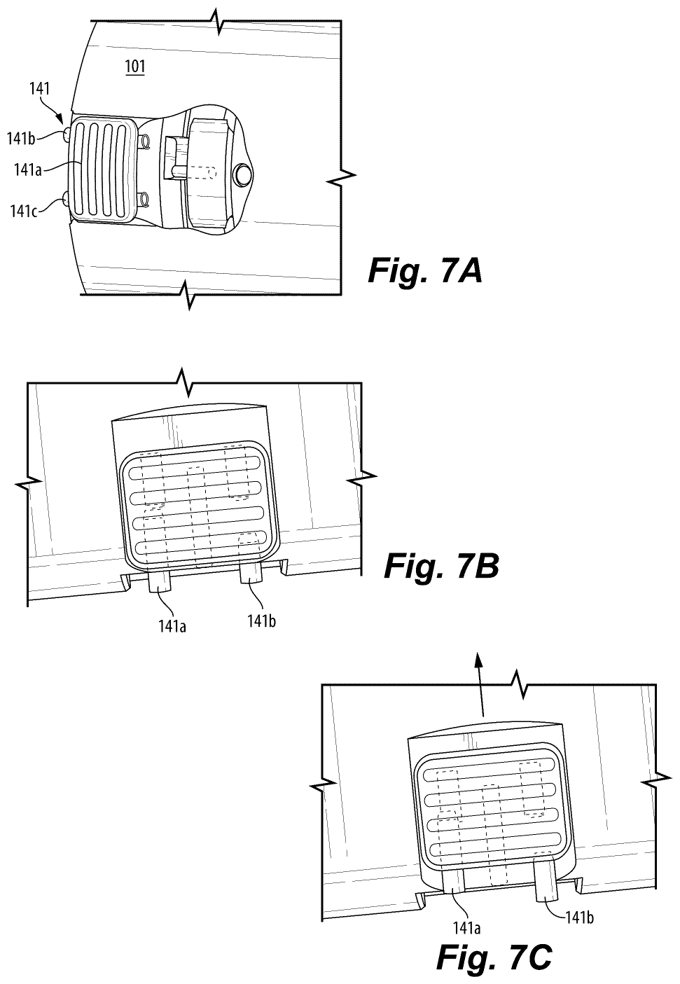

Referring to FIGS. 7A-7C, an embodiment of an instrument release assembly 141 is shown. The assembly 141 can include a slider button 141a configured to slide relative to the outer housing 101 in a slot defined by the outer housing 101, a movable structure 141b (e.g., a pin as shown) that is fixed to the slider button 141a to move with the slider button 141a, and a stationary structure 141c (e.g., a pin as shown) that is fixed relative to the outer housing 101. As shown in FIGS. 7B and 7C, when the slider button 141a is moved proximally, the movable structure 141b moves with it and opens a pathway for a lock tab of the instrument (not shown in FIGS. 7A-7C, described below) to rotate, allowing the proximal hub of the instrument to rotate relative to the outer housing 101 (e.g., the outer housing 101 moving relative to the robotic medical system) to move to the instrument-unrestrained position an allowing axial release of the instrument (e.g., in an emergency).

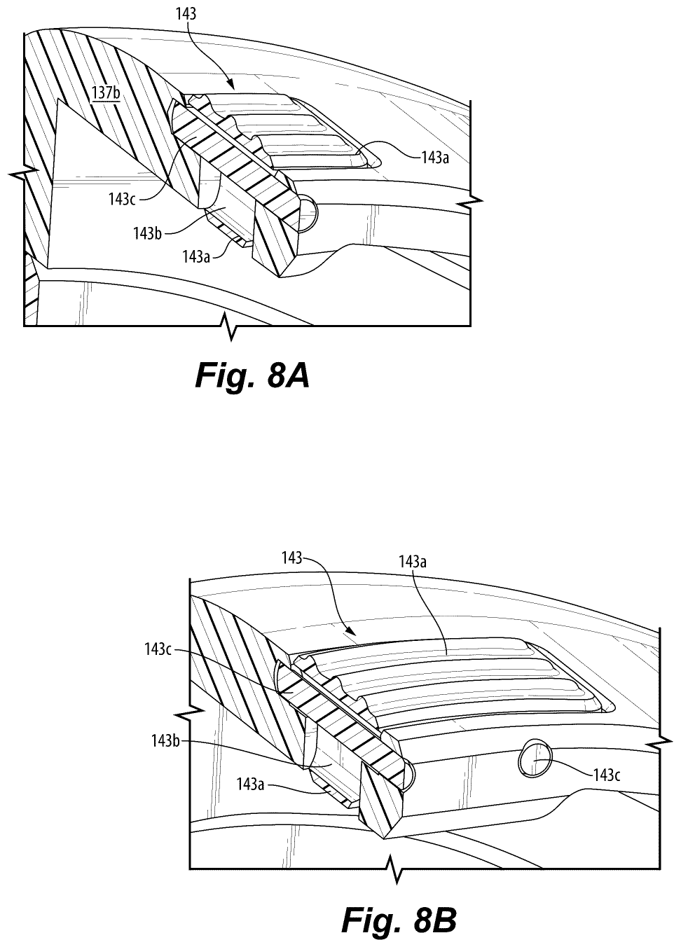

FIG. 8A shows a partial perspective cross-sectional view of a proximal portion of the outer housing of FIG. 1A. An instrument controller attachment assembly 143 is shown, which can have a push button 143a configured to move radially outward guided by axial pins 143c disposed in one or more radial channels 143b of the assembly 145. FIG. 8B shows another partial perspective cross-sectional view of a proximal portion shown in FIG. 8A further illustrating the instrument controller attachment assembly. The push buttons 143a can be urged radially outward by the instrument controller of the robotic medical system such that the instrument controller can clip into the outer housing 101. When ready for removal, a user can press each push button 143a and pull off the outer housing 101.

An example embodiment of a proximal instrument hub 900 configured to interface with the assembly 100 is shown in FIG. 9A. The proximal instrument hub 900 can include one or more hub tabs 901 for interfacing with the instrument alignment tabs 129. For example, the instrument alignment tabs 129 can be inserted into axial channel 903 then rotated in a circumferential aperture 905 to engage behind hub tabs 901 (in the instrument-retained position). The hub can also include a lock tab 907. In certain embodiments, the lock tab 907 can be connected to a lock ring 909 configured to rotate relative to the proximal hub 900. The lock ring 909 can be configured to selectively lock the actuators 911 of the instrument in a locked position (e.g., corresponding to the instrument-unrestrained position), and to release the actuators 911 in an unlocked position (e.g., corresponding to the instrument-retained position). The lock tab 907 is configured to insert between the slider button 141a and the movable structure 141b when axially inserted into the assembly 100. In this regard, rotating the proximal instrument hub 900 relative to the outer housing 101 causes the lock tab to contact one of the structures 141a, 141b and move to the unlocked position to unlock the instrument actuators 911. In an emergency, for example, the slider button 141a can be slid thereby moving the movable structure 141b and allowing reverse rotation of the proximal hub 900 relative to the outer housing 101 which allows removal of the proximal instrument hub 900.

Another example embodiment of a proximal instrument hub 900B configured to interface with the assembly 100 is shown in FIG. 9B. The hub 900B can function similarly to hub 900A, however, can include different insertion structure, for example. For example, the instrument alignment tabs 129 can be inserted into axial channel 903b then rotated in a circumferential annulus 905b to engage behind hub tab wall 901b (in the instrument-retained position). Proximal instrument hub 900B can be otherwise similar to proximal instrument hub 900, for example.

An embodiment of an instrument controller 1000 of a robotic medical system (having a drape around the instrument controller 1000) is shown in FIG. 10A. In certain embodiments, the robotic surgical system can be equipped with one or more instrument controllers 1000 (e.g., configured to connect to a medical device such as a robotically controlled forceps assembly or a videoscope attachable to the instrument controller) for controlling a medical device and/or a steerable overtube (not shown) for performing an endoluminal surgery. FIG. 10B shows an embodiment of the instrument controller interface 1001 removed from the instrument controller 1000. The drape assembly 100 is detachably mountable to the instrument controller, which can be axially slid onto the instrument controller 1000 and the interface 1001 to mount to the robotic medical system. The assemblies 143 can allow clipping of the outer housing 101 to the robotic medical system proximate the instrument controller 1000.

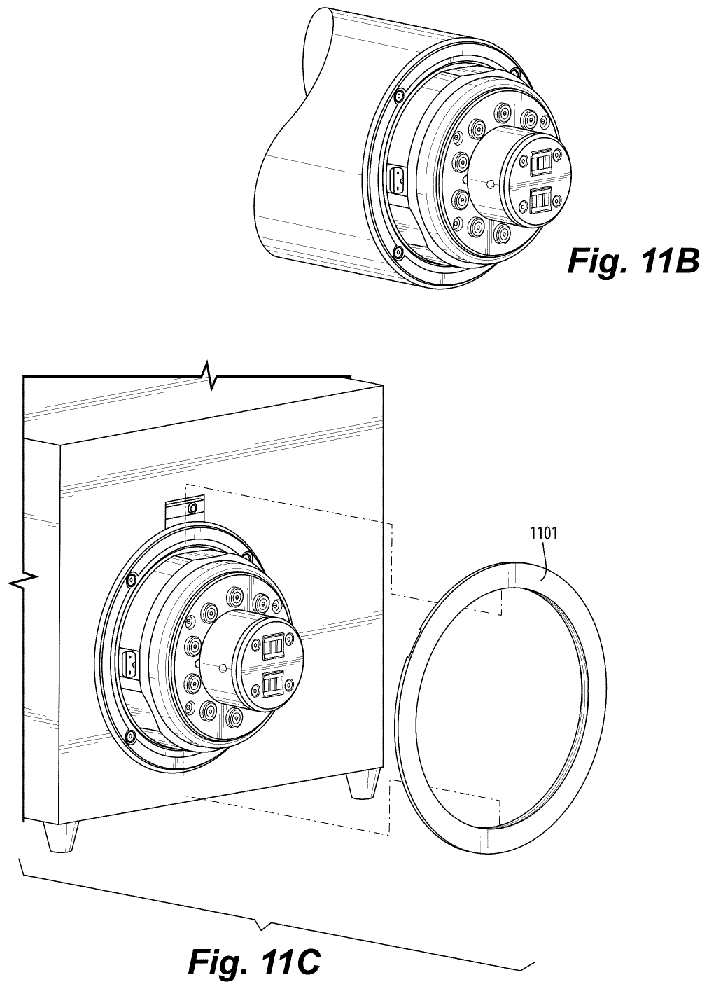

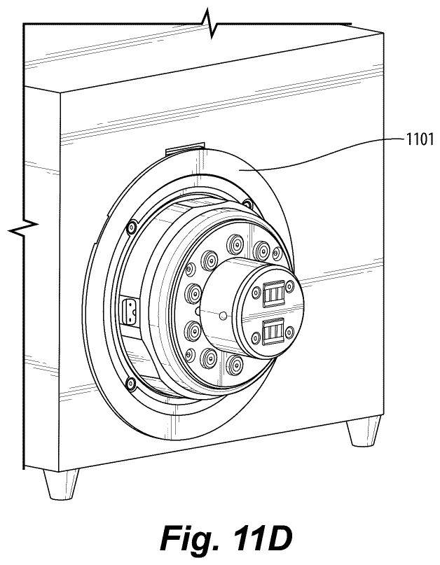

FIG. 11A is a perspective view of an embodiment of an instrument controller in accordance with this disclosure. FIG. 11B is a perspective view of the embodiment of an instrument controller of FIG. 11A. FIG. 11C is a perspective view illustrating a drape ring 1101 relative to the embodiment of FIG. 11A. FIG. 11D is a perspective view illustrating the drape ring of FIG. 11C installed over the embodiment of FIG. 11A. The drape adapter, e.g., as described above can be attached to the instrument controller with a drape ring positioned between the adapter and the controller. For example, FIG. 11C shoes a drape ring 1101, and FIG. 11D shows the drape ring 1101 being placed around the controller. The drape adapter can go over the instrument controller and sandwich the drape ring 1101 between the adapter and the controller. A drape material can be attached to the drape ring 1101, for example.

In accordance with at least one aspect of this disclosure, a robotic surgical instrument controller assembly can include an instrument controller having a mounting interface, an outer housing is detachably mountable to at least a portion of the mounting interface, and an inner housing axially retained to the outer housing and is rotatable relative to outer housing within a limited range to move between an instrument-unrestrained position and an instrument-retained position. The outer housing can be configured to axially install to the instrument controller, and the inner housing is configured to rotationally align one or more actuation elements of the inner housing at a proximal side of the inner housing with one or more corresponding actuators of the instrument controller such that axial movement of the one or more corresponding actuators of the instrument controllers causes respective axial movement of the one or more actuation elements. The assembly can further include a drape ring 1101 detachably mountable to at least a portion of the mounting interface.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the subject disclosure includes reference to certain embodiments, those skilled in the art will readily appreciate that changes and/or modifications may be made thereto without departing from the spirit and scope of the subject disclosure.

What is claimed is:

1. A drape adapter assembly for securing a drape to a portion of a robotic medical system, comprising:
an outer housing configured to connect to the robotic medical system such that the drape is positioned between the outer housing and the portion of the robotic medical system;
an inner housing disposed at least partially within the outer housing and configured to carry one or more moveable actuation elements therein, the inner housing comprising:
a first inner housing portion; and
a second inner housing portion fastened to the first inner housing portion such that the first inner housing portion and the second inner housing portion are axially retained to the outer housing.

2. The assembly of claim 1, wherein the outer housing includes an inner wall and one or more tabs extending from the inner wall, wherein the one or more tabs are configured to be disposed between the first inner housing portion and the second inner housing portion to axially retain the inner housing.

3. The assembly of claim 2, wherein the first inner housing portion is configured to be inserted into the outer housing through a first outer housing opening and the second inner housing portion is configured to be inserted into the outer housing through a second outer housing opening such that the one or more tabs of the outer housing are trapped between the first inner housing portion and the second inner housing portion.

4. The assembly of claim 3, wherein the inner housing defines a trough, wherein the one or more tabs of the outer housing radially extend into the trough.

5. The assembly of claim 4, wherein the inner housing is configured to rotate relative to the outer housing between an instrument-retained position and instrument-unrestrained position.

6. The assembly of claim 5, wherein the one or more tabs include one or more interaction tabs, wherein the inner housing includes one or more interaction components configured to interact with the one or more interaction tabs, wherein the one or more interaction tabs and the one or more interaction components are configured to retain the inner housing in the instrument-unrestrained position and the instrument-retained position, and/or are configured to provide tactile feedback when the inner housing is rotated to the instrument-retained position or instrument-unrestrained position.

7. The assembly of claim 6, wherein the one or more interaction tabs include a first radial detent and a second radial detent, and wherein the one or more interaction components include a biased interaction member biased radially toward the one or more interaction tabs to radially engage the biased interaction member to the one or more interaction tabs and to cause the biased interaction member to enter the first radial detent or the second radial detent when rotationally aligned therewith.

8. The assembly of claim 7, wherein the biased interaction member and the plurality of detents are configured to allow the biased interaction member to be urged out of the plurality of detents with a relative rotation of the outer housing relative to the inner housing.

9. The assembly of claim 6, wherein the one or more tabs include one or more limit tabs, and wherein the inner housing includes one or more limit features configured to interact with the one or more limit tabs to limit a range of rotational motion of the inner housing relative to the outer housing.

10. The assembly of claim 9, wherein the one or more limit tabs or the one or more limit features include a limit pin extending axially therefrom, and the other of the one or more limit tabs and the one or more limit features include an axial slot configured to receive the limit pin, wherein a dimension of the axial slot defines the range of rotation motion of the inner housing relative to the outer housing.

11. The assembly of claim 1, wherein the inner housing defines one or more axial channels configured to receive and allow axial movement of the actuation elements.

12. The assembly of claim 3, wherein the outer housing includes one or more instrument alignment tabs distal of the one or more tabs that extend radially inward from the inner wall of the outer housing, wherein the one or more instrument alignment tabs are configured to align an instrument for insertion in the instrument-unrestrained position, and to retain an instrument to the assembly in the instrument-retained position.

13. The assembly of claim 12, wherein the first inner housing portion includes one or more alignment slots defined in an outer surface thereof configured to align with the instrument alignment tabs to allow axial advancement of the first inner housing portion into the outer housing from the first outer housing opening.

14. The assembly of claim 13, wherein the first inner housing portion includes one or more axial instrument alignment features configured to force rotational alignment of the instrument and the inner housing when the instrument is inserted in the instrument-unrestrained position.

15. The assembly of claim 14, wherein the one or more axial alignment features include one or more axial pins extending distally from the first inner housing portion and configured to insert into an alignment aperture on a proximal side of the instrument.

16. The assembly of claim 1, wherein the inner housing is entirely contained within the outer housing.

17. The assembly of claim 1, wherein the outer housing is generally cylindrical and has proximal portion having a bell shape.

18. The assembly of claim 17, wherein the outer housing comprises one or more alignment lands extending from an inner wall of the proximal portion for alignment with an instrument controller of the robotic medical system.

19. A drape adapter for an instrument controller of a robotic medical system, comprising:
an outer housing;
an inner housing axially retained to the outer housing and configured to rotate relative to outer housing within a limited range to move between an instrument-unrestrained position and an instrument-retained position.

20. The drape adapter of claim 19, wherein the outer housing is configured to axially install to the instrument controller, and the inner housing is configured to rotationally align one or more actuation elements of the inner housing at a proximal side of the inner housing with one or more corresponding actuators of the instrument controller such that axial movement of the one or more corresponding actuators of the instrument controllers causes respective axial movement of the one or more actuation elements.

21. The drape adapter of claim 20, further comprising a drape ring detachably mountable to at least a portion of the mounting interface.

22. The drape adapter of claim 20, wherein the outer housing comprises an inner wall and one or more tabs extending from the inner wall, wherein the one or more tabs are configured to be disposed axially retain the inner housing.

23. The drape adapter of claim 22, wherein the inner housing defines a trough, wherein the one or more tabs of the outer housing radially extend into the trough.

24. The drape adapter of claim 23, wherein the inner housing is rotatable relative to the outer housing between an instrument-retained position and an instrument-unrestrained position.

25. The drape adapter of claim 24, wherein the one or more tabs comprise one or more interaction tabs, wherein the inner housing comprises one or more interaction components configured to interact with the one or more interaction tabs, wherein the one or more interaction tabs and the one or more interaction components are configured to retain the inner housing in the instrument-unrestrained position and the instrument-retained position, and/or are configured to provide tactile feedback when the inner housing is rotated to the instrument-retained position or instrument-unrestrained position.

26. The drape adapter of claim 25, wherein the one or more interaction tabs comprise a first radial detent and a second radial detent, and wherein the one or more interaction components comprise a biased interaction member biased radially toward the one or more interaction tabs to radially engage the biased interaction member to the one or more interaction tabs and to cause the biased interaction member to enter the first radial detent or the second radial detent when rotationally aligned therewith.

27. The drape adapter of claim 26, wherein the biased interaction member and the plurality of detents are configured to allow the biased interaction member to be urged out of the plurality of detents with a relative rotation of the outer housing relative to the inner housing.

28. The drape adapter of claim 25, wherein the one or more tabs comprise one or more limit tabs, and wherein the inner housing comprises one or more limit features configured to interact with the one or more limit tabs to limit a range of rotational motion of the inner housing relative to the outer housing.

29. The drape adapter of claim 28, wherein the one or more limit tabs or the one or more limit features comprise a limit pin extending axially therefrom, and the other of the one or more limit tabs and the one or more limit features comprise an axial slot configured to receive the limit pin, wherein a dimension of the axial slot defines the range of rotation motion of the inner housing relative to the outer housing.

30. The drape adapter of claim 22, wherein the outer housing comprises one or more instrument alignment tabs distal of the one or more tabs that extend radially inward from the inner wall of the outer housing, wherein the one or more instrument alignment tabs are configured to align an instrument for insertion in the instrument-unrestrained position, and to retain an instrument to the assembly in the instrument-retained position.

31. The drape adapter of claim 30, wherein the inner housing comprises one or more alignment slots defined in an outer surface thereof configured to align with the instrument alignment tabs to allow axial advancement of the inner housing into the outer housing.

32. The drape adapter of claim 31, wherein the inner housing further comprises one or more axial instrument alignment features configured to force rotational alignment of the instrument and the inner housing when the instrument is inserted in the instrument-unrestrained position.

33. The drape adapter of claim 32, wherein the one or more axial alignment features comprise one or more axial pins extending distally from the inner housing and configured to insert into an alignment aperture on a proximal side of the instrument.

34. The drape adapter of claim 20, wherein the outer housing comprises one or more alignment lands extending from an inner wall of a proximal portion for alignment with an instrument controller of the robotic medical system.

35. A robotic surgical instrument controller assembly, comprising:
an instrument controller having a mounting interface;
an outer housing detachably mountable to at least a portion of the mounting interface; and
an inner housing axially retained to the outer housing and rotatable relative to the outer housing within a limited range to move between an instrument-unrestrained position and an instrument-retained position.

36. The robotic surgical instrument controller assembly of claim 35, wherein the outer housing is configured to axially install to the instrument controller, and the inner housing is configured to rotationally align one or more actuation elements of the inner housing at a proximal side of the inner housing with one or more corresponding actuators of the instrument controller such that axial movement of the one or more corresponding actuators of the instrument controllers causes respective axial movement of the one or more actuation elements.

37. The robotic surgical instrument controller assembly of claim 36, further comprising a drape ring detachably mountable to at least a portion of the mounting interface.

38. A drape adapter assembly for securing a drape to a portion of a robotic medical system, comprising:
an outer housing configured to connect to the robotic medical system such that the drape is positioned between the outer housing and the portion of the robotic medical system; and
an inner housing disposed at least partially within the outer housing and configured to carry one or more moveable actuation elements thereon, wherein an inner housing axially retained to the outer housing and is rotatable relative to outer housing within a limited range.
